(19) United States
(12) Patent Application Publication
(10) Pub. No.: US 2014/0193851 A1
(43) Pub. Date: Jul. 10, 2014

(54) Detecting Individual Analytes by Means of Magnetic Flow Measurement

(71) Applicant: SIEMENS AKTIENGESELLSCHAFT, Munchen (DE)

(72) Inventors: Oliver Hayden, Herzogenaurach (DE); Michael Johannes Helou, Regensburg (DE); Mathias Reisbeck, Obertraubling (DE); Sandro Francesco Tedde, Erlangen (DE)

(21) Appl. No.: 14/239,077

(22) PCT Filed: Aug. 1, 2012
(86) PCT No.: PCT/EP2012/064986

§ 371 Date: Feb. 14, 2014

(30) Foreign Application Priority Data
Aug. 15, 2011 (DE) 10 2011 080 947.3

(51) Int. Cl. C12Q 1/02

(52) U.S. Cl. CPC C12Q 1/02
USPC 435/29

(57) ABSTRACT

In a magnetic flow measurement, such as flow cytometry, individual analytes are detected in the through-flow. The analytes (e.g., cells) are marked with magnetic labels directly in the medium surrounding the analytes. The analytes are transported through the flow channel of a measuring device including at least one magnetic sensor. Using the magnetic marking of the analytes, the magnetic analyte diameter (r_{mag}) is detected rather than the optical or hydrodynamic size (rₒₚₜ) of the analytes. The analyte diameter is determined by the stray field maximum. The analyte diameter is smaller than the analyte size, such that individual analytes may be detected at high analyte concentrations.

The present patent document is a §371 nationalization of PCT Application Serial Number PCT/EP2012/064986, filed Aug. 1, 2012, designating the United States, which is hereby incorporated by reference in its entirety. This patent document also claims the benefit of DE 10 2011 080 947.3, filed on Aug. 15, 2011, which is also hereby incorporated by reference in its entirety.

### BACKGROUND

The present embodiments relate to the magnetic flow measurement of magnetically marked analytes, and to magnetic flow cytometry.

In magnetic flow cytometry, two approaches are used for single-cell detection. In single-cell detection, the problem of clear separation of two cells directly following one another is overcome in the following way.

As described in Loureiro et al., Journal of Applied Physics, 2011, 109, 07B311, superparamagnetically marked cell analytes are detected by a magnetoresistive sensor. The marked cells are not very highly concentrated, as enrichment of the cells is not employed, but this also leads to a very low detection rate, i.e., only a small percentage of the marked cells are registered by the magnetoresistive sensor.

Alternatively, operation is implemented with dilute samples. By reduction of the concentration of a cell suspension in combination with enrichment of the magnetically marked cells, the spacing is increased and the cells are guided individually over the sensor, although the measurement time may be undesirably lengthened.

### SUMMARY AND DESCRIPTION

The present embodiments may obviate one or more of the drawbacks or limitations in the related art. For example, single-cell detection having a high detection rate and a short measurement time is provided.

A method for magnetic flow measurement of an analyte includes implementing a magnetic marking of analytes in a sample. A flow of the analytes, which guides the analytes over a sensor arrangement, is then generated. The flow of the analytes is guided at least over a magnetoresistive component. A gradient magnetic field is also generated, via which the marked analytes are enriched over the magnetoresistive component, and a homogeneous magnetic field is generated. The homogenous magnetic field is oriented (e.g., extends) with respect to the magnetoresistive component such that the homogeneous magnetic field is not detected by the magnetoresistive component. The detection of individual marked analytes is implemented via the sensor arrangement having the at least one magnetoresistive component. The magnetic marking is implemented such that, in the homogeneous magnetic field, the marked analytes each induce a stray magnetic field having detectable maxima that lie at a distance from the analyte center. The distance is less than the analyte hydrodynamic radius.

The gradient magnetic field and of the homogeneous magnetic field are generated by a single magnetic unit that provides a double function. At a further distance from the magnetic unit, the magnetic unit generates the gradient field for enrichment of the magnetically marked analytes. Close to the magnetic unit, however, the magnetic field lines extend homogeneously. The magnetic unit may be arranged with respect to the sensor, i.e. the magnetoresistive component, such that the homogeneous field is oriented (e.g., extends) in a direction in which the sensor is not sensitive. As a result, for example, the homogeneous magnetic field is oriented (e.g., extends) in the z direction while the sensor is sensitive in the x direction perpendicular to the z direction.

The stray field induced in the homogeneous magnetic field by the magnetic marking of an analyte is detected by the magnetoresistive component. The x component of this stray field is measured. The x direction is defined as the flow direction, i.e., the direction of the stray field parallel to the surface of the magnetoresistive component. The detectable stray field maxima thus establish a distance from the analyte center. The distance is referred to herein as the magnetic radius. As a result of the magnetic marking of analytes (e.g., cell analytes or beads), the analytes may have a magnetic diameter less than the optical or hydrodynamic diameter. The maximum stray field in the x direction may thus be disposed within the contour of the analyte. In this manner, the detection of two cells immediately following one another may be achieved separately as two individual events by detection of the x component of the stray field with, for example, a magnetoresistive component sensitive in this horizontal x direction.

Analytes may thus be detected individually, even with high cell concentrations in which the analytes are present at the shortest possible spacing. So-called individual events may be resolved. Through suitable magnetic marking, the stray field of a marked analyte is thus influenced, e.g., in a vertical external magnetic field, such that a high detection rate of the single-analyte magnetic detection is provided.

The sensor arrangement may include at least one magnetoresistive component. Alternatively, a plurality of magnetoresistive components, such as individual resistors, may be included. For example, the sensor arrangement includes individual magnetoresistive resistors interconnected in, for instance, in a Wheatstone measurement bridge. As described in the patent application DE 10 2010 040 391.1, characteristic signal profiles may be generated by the Wheatstone measurement bridge.

In one embodiment of the method, the magnetic marking is implemented with magnetic nanobeads, e.g., superparamagnetic nanobeads. The nanobeads may have, for example, a hydrodynamic diameter of between 10 nm and 500 nm. Depending on the analytes to be marked, e.g., depending on the cell type, the surface and/or epitope number of the nanobeads determines the size and type of the marking Small nanobeads with a diameter of between 10 nm and 500 nm may provide occupation densities on the analyte surface of between 10% and 90%, which achieve displacement of the stray field maximum into the interior of the analyte. For instance, an analyte, e.g., a cell, is marked such that the maximum of the x component of the stray field lies at a distance of between 50% and 90% of the cell radius away from the cell center.

In another embodiment of the method, the magnetic marking is implemented with nanobeads that include the material magnetite or maghemite. For example, the nanobeads used for the marking include a material having a saturation magnetization that lies approximately between 80 and 90 emu/g.

The material proportion of the nanobeads may be selected such that the magnetization of the magnetic beads is approximately between 10 and 60 (A·m²)/kg.

In an example with cells having an average diameter of 12 μm, a stray field maximum in the x direction at a distance of, on average, 4 μm from the cell center may be induced with suitable magnetic marking. This reduced magnetic radius may allow the cells marked in this manner to be detected individually in a vertical external magnetic field, even if the cells flow over the sensor arrangement in direct contact with one another.

In one embodiment of the method, the individual marked analytes are enriched over the magnetoresistive component via the gradient magnetic field. As a result, the analytes are locally present in a high concentration. Starting from sample concentrations of from 0.1 to 10⁴ analytes per microliter, the concentration is increased by the enrichment to between 100 times and 10,000 times. A very high detection rate may be achieved, because only an exceedingly small proportion of the analytes fail to pass close enough by the sensor to be detected thereby. At the same time, the high concentration, in which the individual analytes may be in direct contact with one another, does not result in the analytes being counted as a single event. Rather, as a result of the reduced magnetic radius, which is ultimately detected by the magnetoresistive sensor arrangement, the analytes may be separated even in the event of direct contact of the cells. The measurement system thus simultaneously provides a high detection rate, even when two cells directly follow one another, as well as a measurement on a suspension in which the analytes are present in a very high concentration. If the magnetic marking results in the stray field maximum being disposed inside the cell, then two marked analytes immediately following one another may be measured as two individual events. The individual analytes may thus flow over the magnetoresistive component in direct contact with one another.

Magnetophoretic enrichment of the magnetically marked analytes may be implemented for enrichment of the magnetically marked analytes in addition to the gradient magnetic field. The gradient magnetic field may be induced by a permanent magnet. Magnetophoretic enrichment is described in the patent application DE 10 2009 0477 801.9, which discloses a system for controlled transport of magnetically marked cells in a flowing medium for magnetic flow cytometry. In one embodiment of the method, the flow speed is adjusted such that the analytes are guided over the magnetoresistive component with a constant speed. The flow speed may be adjusted such that the analytes, e.g., cells, roll over the magnetoresistive component. In this case, upon contact with the channel wall on or in which the magnetoresistive component may be arranged, the analytes are set in rotation and roll along the wall and therefore over the magnetoresistive component. The magnetoresistive component or the plurality of magnetoresistive bridge elements, may be giant magnetoresistance (GMR) sensors.

### BRIEF DESCRIPTION OF THE DRAWINGS

FIG. 1 is a schematic, side view of a magnetic unit in accordance with one embodiment.

FIG. 2 is a graphical plot of a distribution function in accordance with one embodiment.

FIG. 3 is a schematic view of a permanent magnet and a homogenous magnetic field generated by the permanent magnet in accordance with one embodiment.

FIG. 4 is a schematic view of a measurement structure of a microfluidic system in accordance with one embodiment.

FIG. 5 shows a schematic view and graphical plot of a magnetoresistive signal in accordance with one embodiment.

### DETAILED DESCRIPTION

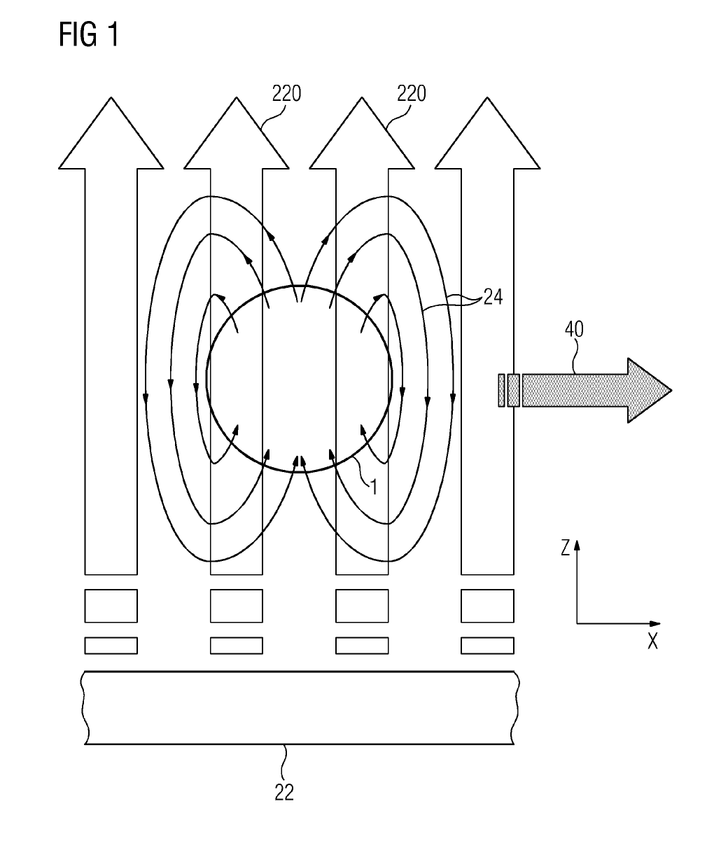

FIG. 1 shows a side view of the magnetic unit 22 for generation of the gradient field and of the homogeneous magnetic field 220, which is indicated by arrows perpendicular to the magnetic unit 22. The magnetic marking of the analyte 1 induces a stray magnetic field 24 of the analyte. The magnetic field line profile of the stray magnetic field 24 is shown around the analyte 1. The analyte 1 is represented as a circle in cross section. The arrow 40, which points from left to right in FIG. 1, indicates the flow direction of the analyte 1. The magnetic unit 22 is located, for example, below a flow channel for an analyte sample, e.g., a cell sample.

The magnetic unit 22 may have a double function. For example, the gradient field generated by the external magnet 22 attracts the superparamagnetically marked cells 1 onto the sensor surface 20. There, the cells 1 are stochastically distributed. In the flow 40, the cells 1 are guided magnetophoretically, e.g., with the aid of nickel strips, over the magnetoresistive sensors 20. Directly over the sensor 20, an essentially homogeneous field 220 is generated, which, as shown in FIG. 1, extends only in the z direction. Thus, the sensor 20 does not see a vertical field 220 because the sensor 20 is sensitive only in the x direction. FIG. 1 thus shows, for example, a superparamagnetically marked cell 1 that distorts the field 220 in the vicinity of the superparamagnetically marked cell 1. The x component of the stray field 24 is the field which is detected by the sensor 20. The inhomogeneity of the magnet 22 that generates the external field is thus utilized in the device. The magnet 22 may be, for example, an NdFeB magnet. The homogeneous region 220 adjacent, e.g., close, to the magnet 22 varies, depending on the quality of the magnet 22. The homogenous region 220 is placed below the sensor 20. The gradient field for the enrichment is then provided by the inhomogeneity of the magnetic field outside the homogeneous region 220.

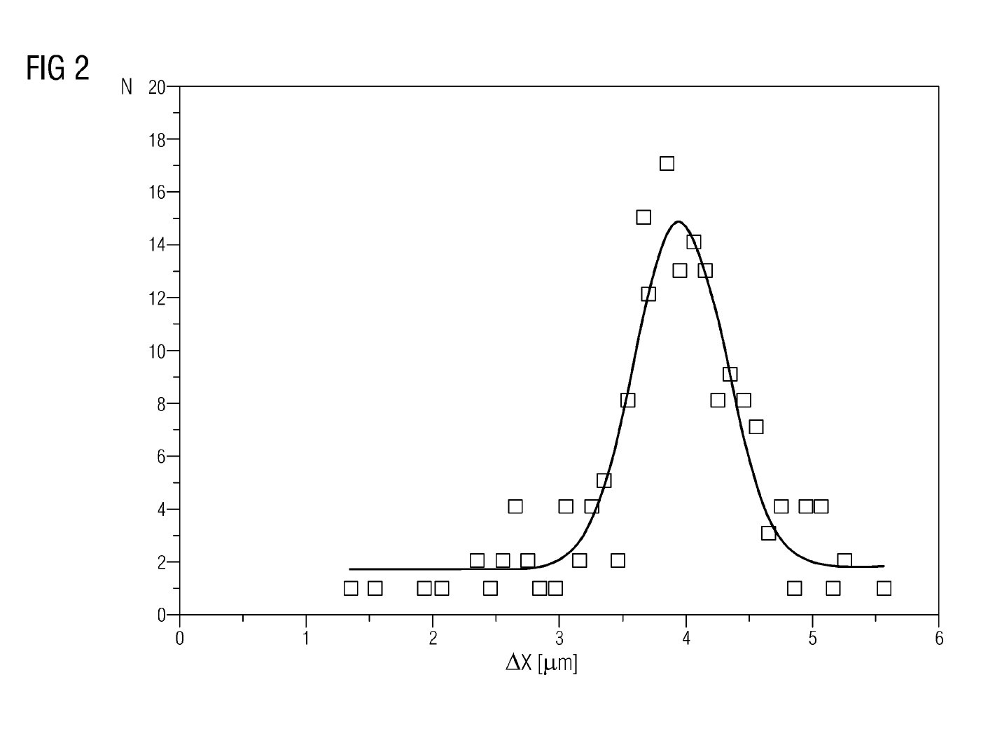

FIG. 2 shows a diagram of a distribution function N and measurement points indicated by squares. The number of analytes 1, e.g., cells, having a stray field 24 with a maximum in the x direction are measured as a distance Δx from the center of the analyte. The x direction is detected by the sensors. The distance Δx is indicated in μm.

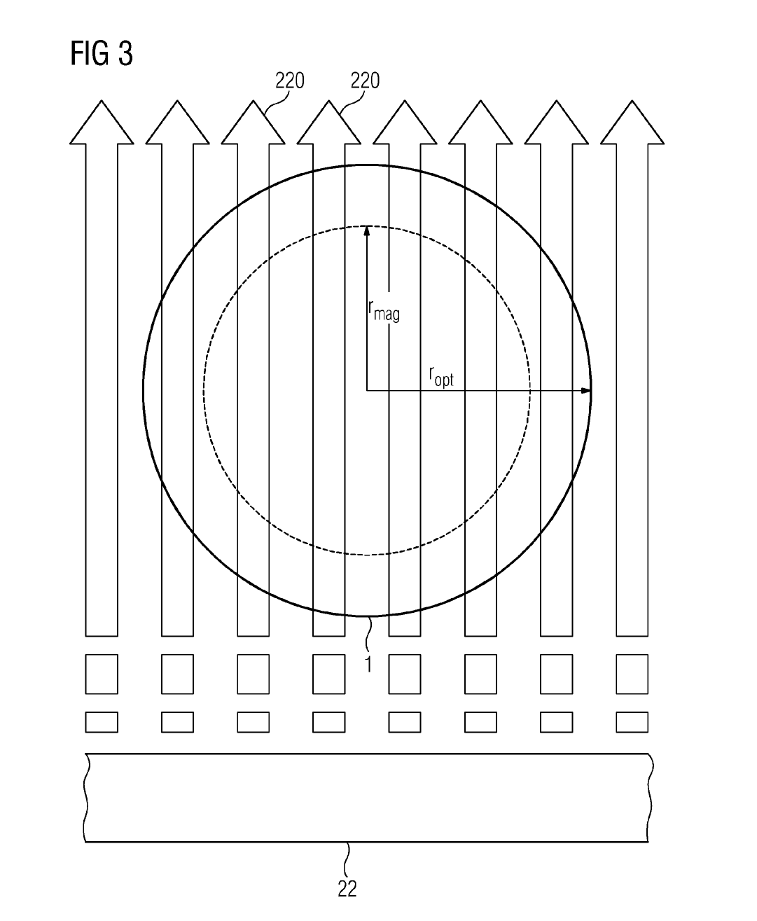

FIG. 3 shows a representation of the permanent magnet 22 and of the homogeneous magnetic field 220 generated by the permanent magnet 22. The cell 1 has an optical or hydrodynamic diameter rₒₚₜ, and also a so-called magnetic diameter r_{mag}, which may be less than the optical diameter rₒₚₜ, (e.g., the magnetic diameter r_{mag} lies inside the cell 1). The smaller diameter results from the maximum stray field component in the x direction, which is detected by the magnetic sensors 20, being disposed at a position located inside the cell 1. Thus, even if the magnetic markers are placed on the surface of the cell 1, the stray field 24 generated by the magnetic marking is disposed both outside and inside the cell 1, as does the maximum of the stray field 24 in the x direction.

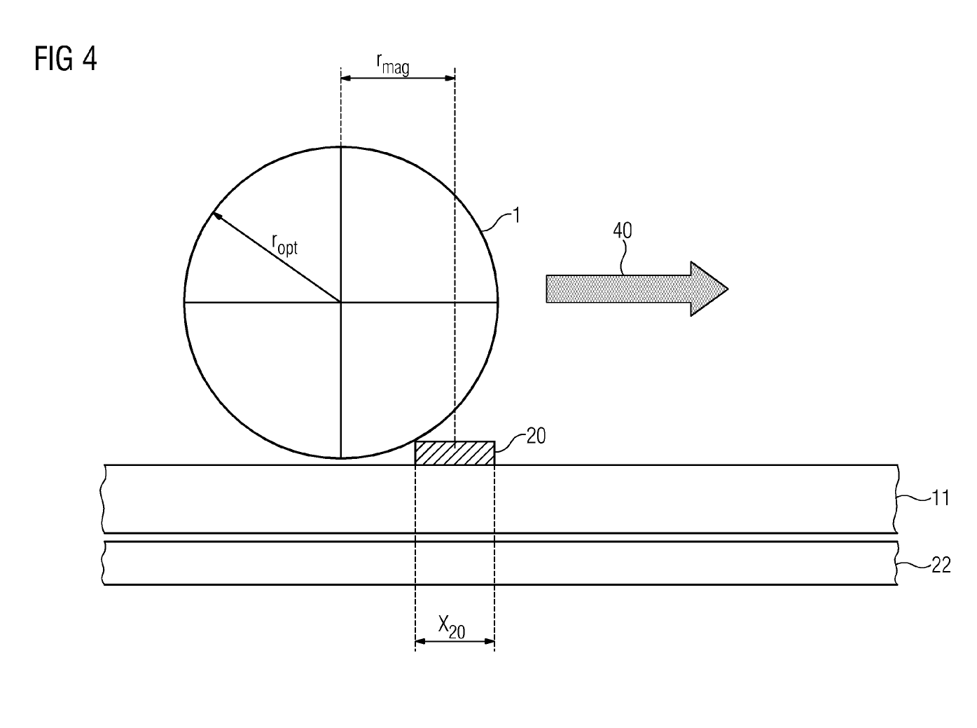

FIG. 4 schematically shows the measurement structure, such as a portion of a microfluidic system having a flow channel. The channel bottom 11 includes at least one magnetic sensor 20. The magnetic unit 22 for generation of the gradient field and the homogeneous magnetic field 220 is disposed, e.g., fitted, below the channel bottom 11. The magnetic sensor 20 may have a length x₂₀ in the flow direction 40. The first maximum measurement excursion, however, occurs not at the moment when the cell 1 reaches the sensor 20 with its optical or hydrodynamic diameter rₒₚₜ, but, as indicated by a dashed line, only when the maximum of the x component of the stray magnetic field 24 extending through the cell 1 passes over the edge of the sensor 20. This position marks the magnetic radius r_{mag}, which may be less than the optical radius rₒₚₜ of the cell 1. Once the cell 1 has passed over the magnetic sensor 20, a second maximum measurement excursion is registered in the other magnetic field direction.

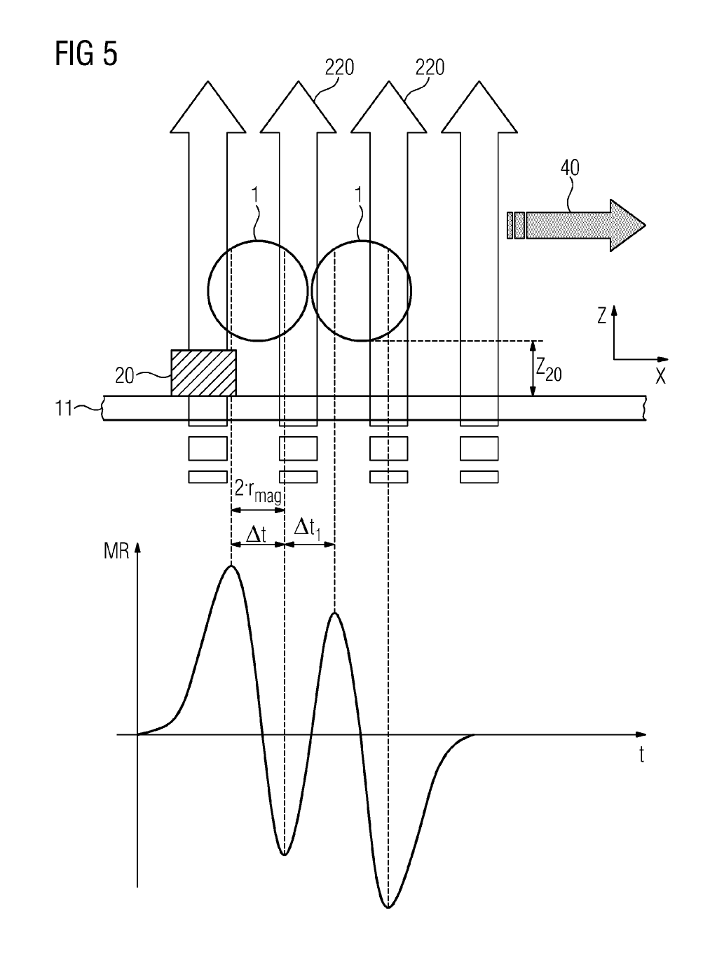

FIG. 5 shows the magnetoresistive signal, recorded over a period of time, of a plurality of cells 1 following one another. In cases in which the magnetic diameter r_{mag} is the same as the optical or actual cell diameter rₒₚₜ of the cell 1, when two adjacent cells 1 pass over, as shown at the top in FIG. 5, a positive first measurement event (e.g., excursion) caused by the first cell 1 passing over the sensor 20, and a negative second measurement event (e.g., excursion) caused by the end of the second cell 1, are detected. Yet because the magnetic diameter is disposed inside the cell 1, the measurement events (e.g., excursions), which are related to the maximum of the x component of the stray field 24 of a cell 1, are separated sufficiently far from one another, Δt₁. As a result, each cell 1 induces a full measurement signal of two measurement events (e.g., excursions), as shown in the lower diagram of FIG. 5. The time difference Δt of the measurement excursions of a cell signal is correlated with the magnetic diameter 2·r_{mag} of a magnetically marked cell 1. FIG. 5 also shows the homogeneous magnetic field 220 in the z direction. The distance of the cells 1 from the channel bottom 11 is marked by z₂₀. The cells 1 pass over the magnetic sensor 20 in the flow direction 40.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for magnetic flow measurement of an analyte, the method comprising:
magnetic marking of analytes in a sample;
generating a flow of the analytes over a sensor arrangement, the flow of the analytes being guided at least over a magnetoresistive component;
generating a gradient magnetic field to enrich the marked analytes over the magnetoresistive component, and a homogeneous magnetic field, the homogeneous magnetic field and the magnetoresistive component being arranged with respect to one another such that the homogeneous magnetic field is not detected by the magnetoresistive component; and
detecting the marked analytes individually;
wherein the magnetic marking is implemented such that each marked analyte of the analytes has a stray magnetic field with a maxima detectable by the magnetoresistive component and disposed at a distance from a center of the analyte less than a hydrodynamic radius of the analyte.

2. The method of claim 1, wherein the magnetic marking is implemented with magnetic nanobeads.

3. The method of claim 1, wherein the magnetic marking is implemented with nanobeads having a hydrodynamic diameter between 10 nm and 500 nm.

4. The method of claim 1, wherein the magnetic marking is implemented with nanobeads comprising magnetite or maghemite.

5. The method as of claim 1, wherein the magnetic marking is implemented with nanobeads having a magnetization of between 10 and 60 (A·m2)/kg.

6. The method of claim 1, further comprising enriching the marked analytes over the magnetoresistive component via the gradient magnetic field, such that the marked analytes are locally present in a concentration increased from sample concentrations by a factor of between 100 and 10,000.

7. The method of claim 1, wherein the marked analytes are in direct contact with one another when flowing over the magnetoresistive component.

8. The method of claim 1, further comprising adjusting a speed of the flow such that the analytes are guided over the magnetoresistive component with a constant speed.

9. The method of claim 1, wherein the magnetic marking is implemented with superparamagnetic nanobeads.

10. The method of claim 9, wherein the superparamagnetic nanobeads have a hydrodynamic diameter between 10 nm and 500 nm.

11. The method of claim 9, wherein the superparamagnetic nanobeads comprise magnetite or maghemite.

12. The method of claim 9, wherein the superparamagnetic nanobeads have a magnetization of between 10 and 60 (A·m2)/kg.

13. The method of claim 1, further comprising enriching the marked analytes over the magnetoresistive component via the gradient magnetic field, such that the marked analytes are locally present in a concentration increased from sample concentrations of 0.1 to 104 analytes per microliter by a factor of between 100 and 10,000.

14. The method of claim 1, further comprising adjusting a speed of the flow such that the analytes roll over the magnetoresistive component with a constant speed.

15. A method for magnetic flow measurement of an analyte comprising:
magnetic marking of analytes in a sample with magnetic nanobeads;
generating a flow of the analytes over a sensor arrangement, the flow of the analytes being guided at least over a magnetoresistive component;
enriching the marked analytes over the magnetoresistive component with a gradient magnetic field;
generating a homogeneous magnetic field, the homogeneous magnetic field and the magnetoresistive component being arranged with respect to one another such that the homogeneous magnetic field is not detected by the magnetoresistive component; and
detecting the marked analytes individually;
wherein the magnetic marking is implemented such that each marked analyte of the analytes has a stray magnetic field with a maxima detectable by the magnetoresistive component and disposed at a distance from a center of the analyte less than a hydrodynamic radius of the analyte.

16. The method of claim 15, further comprising enriching the marked analytes over the magnetoresistive component via the gradient magnetic field, such that the marked analytes are locally present in a concentration increased by a factor of between 100 and 10,000.

17. The method of claim 15, further comprising enriching the marked analytes over the magnetoresistive component via the gradient magnetic field, such that the marked analytes are locally present in a concentration increased from sample concentrations of 0.1 to 104 analytes per microliter by a factor of between 100 and 10,000.

18. The method of claim 15, wherein the marked analytes are in direct contact with one another when flowing over the magnetoresistive component.

19. The method of claim 15, further comprising adjusting a speed of the flow such that the analytes are guided over the magnetoresistive component with a constant speed.

20. The method of claim 15, further comprising adjusting a speed of the flow such that the analytes roll over the magnetoresistive component with a constant speed.
